(19) United States
(12) Patent Application Publication
(10) Pub. No.: US 2026/0241084 A1
(43) Pub. Date: Aug. 20, 2026

(54) EXTRACORPOREAL BLOOD TREATMENT TUBING SET AND APPARATUS

(71) Applicant: GAMBRO LUNDIA AB, Lund (SE)

(72) Inventors: Wei ZHU, Suzhou, Jiangsu (CN); Jiaolong LV, Suzhou, Jiangsu (CN); Yue Cheng CUI, Suzhou, Jiangsu (CN); Juanhua LIU, Shanghai (CN)

(21) Appl. No.: 19/489,525

(22) PCT Filed: Jun. 27, 2023
(86) PCT No.: PCT/EP2023/067492

§ 371 Date: Dec. 2, 2025

(30) Foreign Application Priority Data
Jun. 2, 2023 (CN) 202310652928.7

(51) Int. Cl. A61M 1/14; A61M 1/36; A61M 39/00; A61M 39/10

(52) U.S. Cl. CPC A61M 1/153; A61M 1/3603; A61M 1/36223; A61M 1/3623; A61M 39/10; A61M 2039/0009; A61M 2039/1027; A61M 2202/0021; A61M 2202/0413; A61M 2205/127; A61M 2205/3331; A61M 2205/3368; A61M 2205/50; A61M 2205/583

(57) ABSTRACT

An extracorporeal blood treatment apparatus (1) comprising a fluid circuit (6) coupled to a blood treatment unit (2), a fluid pump (9) to determine a fluid flow inside the fluid circuit (6), and a fluid temperature control device (15). A fluid warming/cooling bag (24) coupled to the fluid circuit (6) comprises a first fluid conduit (25) configured for putting a first inlet port (25a) in fluid communication with a first outlet port (25b) and a second fluid conduit (26) configured for putting a second inlet port (26a) in fluid communication with a second outlet port (26b), the first and the second fluid conduit (25, 26) being fluidically separated from each other. When the fluid warming/cooling bag (24) is accommodated in the fluid temperature control device (15), the latter simultaneously determining a heat exchange with the two fluids inside the first and second fluid conduits (25, 26) of the fluid warming/cooling bag (24).

### TECHNICAL FIELD

The present disclosure relates generally to an extracorporeal blood treatment apparatus, such as a dialysis treatment apparatus, a corresponding extracorporeal blood treatment tubing set, a corresponding fluid warming/cooling bag and a relative packaging.

The extracorporeal blood treatment apparatus includes a temperature control device that is configured to regulate the temperature of medical fluids, for example blood or treatment fluid or infusion solution/s, in combination with a corresponding fluid warming/cooling bag. The fluid warming/cooling bag is in particular inserted into the temperature control device and the temperature is regulated by means of a heat exchange region of the temperature control device which provides the fluid temperature regulation of the fluid/s into fluid warming/cooling bag.

### BACKGROUND

Extracorporeal blood treatment involves removing blood from a patient, treating the blood externally to the patient, and returning the treated blood to the patient. Extracorporeal blood treatment is typically used to extract undesirable matter or molecules from the patient's blood and add desirable matter or molecules to the blood. Extracorporeal blood treatment is used with patients unable to effectively remove matter from their blood, such as when a patient has suffered temporary or permanent kidney failure. These patients and other patients may undergo extracorporeal blood treatment to add or remove matter to their blood, to maintain an acid/base balance or to remove excess body fluids, for example.

Extracorporeal blood treatment is typically accomplished by removing the blood from the patient in e.g. a continuous flow, introducing the blood into a primary chamber, also referred to as blood chamber, of a treatment unit (such as a dialyzer or an hemofilter) where the blood is allowed to flow past a semipermeable membrane. The semipermeable membrane selectively allows matter in the blood to cross the membrane from the primary chamber into a secondary chamber and also selectively allows matter in the secondary chamber to cross the membrane into the blood in the primary chamber, depending on the type of treatment.

A number of different types of extracorporeal blood treatments may be performed. In an ultrafiltration (UF) treatment, undesirable matter is removed from the blood by convection across the membrane into the secondary chamber. In a hemofiltration (HF) treatment, the blood flows past the semipermeable membrane as in UF and desirable matter is added to the blood, typically by dispensing a fluid into the blood either before and/or after it passes through the treatment unit and before it is returned to the patient. In a hemodialysis (HD) treatment, a secondary fluid containing desirable matter is introduced into the secondary chamber of the treatment unit. Undesirable matter from the blood crosses the semipermeable membrane into the secondary fluid and desirable matter from the secondary fluid may cross the membrane into the blood by diffusion. In a hemodiafiltration (HDF) treatment, blood and secondary fluid exchange matter as in HD, and, in addition, matter is added to the blood, typically by dispensing a fluid into the treated blood before its return to the patient as in HF.

During extracorporeal blood treatment therapies, the patient may lose significant amount of heat due to fluid exchange by diffusion or convection, and due to heat lost to the atmosphere. As extracorporeal blood treatments may last from several hours up to several days, the patient is put at risk of hypothermia in case no preventive measures are taken. This risk is, for example, present both in the case of relatively short treatments with high volume exchange, like chronic HD, and in the case of low volume but continuous therapies like continuous renal replacement therapy (CRRT).

Therefore, in medical applications such as extracorporeal blood treatment it is often desired to regulate the temperature of medical fluids, for example in cases where the individual temperature of a medical fluid is important for particular chemical or biological reactions to take place, or for treatment to be performed under optimal conditions. Other cases include returning blood to a patient that has been withdrawn for extracorporeal treatment (e.g. dialysis, hemofiltration, hemodiafiltration).

In many cases, the flow rate of medical fluid varies over time and depends on a number of factors including treatment type, patient properties and condition, topology of the extracorporeal circuit, etc. Regulating the temperature under these varying conditions requires a system facilitating reliable treatment and handling of medical fluids.

Document EP3791900 shows and discloses a container for warming fluids. The container comprises an inlet port, an outlet port, and one fluid conduit configured for putting the inlet port in fluid communication with the outlet port. The fluid conduit comprises one or more deflection sections and has a non-constant maximum width in a direction of fluid flow through the fluid conduit. The container disclosed in EP3791900 is able to be used with a temperature control device in order to regulate a temperature of the blood returned to the patient to a desired temperature region, for example around 37° C. The temperature control device is generally designed to regulate the temperature of (e.g., to warm) medical fluids such as blood or dialysate/replacement liquid and it may be employed to warm medical fluids other than blood intended to be returned or fluids intended to be supplied to the body of a patient, for example when regulating the temperature of treatment solution entering filtration unit. In other terms, the fluid warming/cooling bag may be differently connected also to the dialysis line.

Also document WO2019/219544 shows and discloses both a fluid warming/cooling bag and a fluid temperature control device for an extracorporeal blood treatment apparatus arranged to accommodate the fluid warming/cooling bag to regulate the temperature of a medical fluid flowing in it. Similarly to the fluid warming/cooling bag of document EP3791900, the fluid warming/cooling bag of document WO2019/219544 comprises an inlet port, an outlet port, and one fluid conduit configured for putting the inlet port in fluid communication with the outlet port. The fluid temperature control device comprises a casing delimiting a heat exchange seat configured to accommodate the fluid warming/cooling bag which is connected or is configured to be connected to an extracorporeal blood treatment apparatus.

Both document EP3791900 and document WO2019/219544 disclose an example of an extracorporeal blood treatment apparatus provided with the corresponding temperature control device for regulating the temperature of a medical fluid flowing through the corresponding fluid conduit of the corresponding fluid warming/cooling bag in use.

### SUMMARY

The present disclosure sets forth an extracorporeal blood treatment apparatus, such as a dialysis treatment apparatus, a corresponding extracorporeal blood treatment tubing set (configured to be coupled to the extracorporeal blood treatment apparatus in detail to perform a dialysis treatment), a corresponding fluid warming/cooling bag and a relative packaging.

In light of the disclosure set forth herein, and without limiting the disclosure in any way, in a 1^{st} independent aspect, which may be combined with any other aspect or portion thereof described herein, an extracorporeal blood treatment apparatus (1) comprises:
a blood treatment unit (2);
a fluid circuit (6) coupled to the blood treatment unit (2);
a fluid pump (9) coupled to the fluid circuit (6) to determine a fluid flow inside the fluid circuit (6);
a fluid temperature control device (15) coupled to the fluid circuit (6) and comprising:
   a receptacle (17);
   a heat exchange region (18);
a fluid warming/cooling bag (24) configured to be accommodated to the receptacle (17) of the fluid temperature control device (15), the fluid warming/cooling bag (24) being coupled to the fluid circuit (6) and comprising:
   a first inlet port (25a);
   a first outlet port (25b);
   a first fluid conduit (25) configured for putting the first inlet port (25a) in fluid communication with the first outlet port (25b);
   a second inlet port (26a);
   a second outlet port (26b),
   a second fluid conduit (26) configured for putting the second inlet port (26a) in fluid communication with the second outlet port (26b), the first fluid conduit (25) and the second fluid conduit (26) being fluidically separated from each other;
      wherein, when the fluid warming/cooling bag (24) is accommodated in the receptacle (17), the fluid temperature control device (15) is configured to determine a heat exchange with at least one fluid inside one of the first and second fluid conduits (25, 26) of the warming/cooling bag (24) at the heat exchange region (18), optionally for simultaneously determining a heat exchange with the at least two (separate) fluids inside the first and second fluid conduits (25, 26) of the fluid warming/cooling bag (24).

In a 2^{nd} independent aspect, which may be combined with any other aspect or portion thereof described herein, an extracorporeal blood treatment apparatus (1) comprises:
a blood treatment unit (2);
fluid circuit (6) coupled to the blood treatment unit (2), the fluid circuit (6) including:
   an extracorporeal blood circuit (7) with a blood withdrawal line (7a) connected to an inlet port (3a) of the blood treatment unit (2) and a blood return line (7b) connected to an outlet port (3b) of the blood treatment unit (2);
   a treatment fluid circuit (8) operatively connected to the extracorporeal blood circuit (7) and/or to the blood treatment unit (2);
a fluid pump (9) coupled to the fluid circuit (6) to determine a fluid flow inside the fluid circuit (6);
a controller (35) controlling the fluid pump (9);
a fluid temperature control device (15) coupled to the fluid circuit (6) and comprising:
   a receptacle (17);
   a heat exchange region (18);
a fluid warming/cooling bag (24) configured to be accommodated to the receptacle (17) of the fluid temperature control device (15), the fluid warming/cooling bag (24) being coupled to the fluid circuit (6) and comprising:
   a first inlet port (25a);
   a first outlet port (25b);
   a first fluid conduit (25) configured for putting the first inlet port (25a) in fluid communication with the first outlet port (25b);
   a second inlet port (26a);
   a second outlet port (26b),
   a second fluid conduit (26) configured for putting the second inlet port (26a) in fluid communication with the second outlet port (26b), the first fluid conduit (25) and the second fluid conduit (26) being fluidically separated from each other;
      wherein, when the fluid warming/cooling bag (24) is accommodated in the receptacle (17), the fluid temperature control device (15) is configured to determine a heat exchange with at least one fluid inside one of the first and second fluid conduits (25, 26) of the warming/cooling bag (26) through the corresponding fluid conduit (25, 26), optionally for simultaneously determining a heat exchange with the at least two (separate) fluids inside the first and second fluid conduits (25, 26) of the warming/cooling bag (24).
      The controller (35) of the apparatus (1) may also control the fluid temperature control device (15); alternatively, the fluid temperature control device (15) may be controlled by a different or separate control unit.

In a 3^{rd} independent aspect, which may be combined with any other aspect or portion thereof described herein, an extracorporeal blood treatment tubing set (2, 6, 7, 8) for an extracorporeal blood treatment apparatus (1) comprises:
a blood treatment unit (2);
a fluid circuit (6) coupled to the blood treatment unit (2), the fluid circuit (6) including:
   an extracorporeal blood circuit (7) with a blood withdrawal line (7a) connected to an inlet port (3a) of the blood treatment unit (2) and a blood return line (7b) connected to an outlet port (3b) of the blood treatment unit (2);
   a treatment fluid circuit (8) operatively connected to the extracorporeal blood circuit (2) and/or to the blood treatment unit (2);
a fluid warming/cooling bag (24) coupled to the fluid circuit (6) and comprising:
   a first inlet port (25a);
   a first outlet port (25b);
   a first fluid conduit (25) configured for putting the first inlet port (25a) in fluid communication with the first outlet port (25b);
   a second inlet port (26a);
   a second outlet port (26b),
   a second fluid conduit (26) configured for putting the second inlet port (26a) in fluid communication with the second outlet port (26b), the first fluid conduit (25) and the second fluid conduit (26) being fluidically separated from each other.

In a 4^{th} independent aspect, which may be combined with any other aspect or portion thereof described herein, a fluid warming/cooling bag (24) of an extracorporeal blood treatment tubing set (2, 6, 7, 8) for an extracorporeal blood treatment apparatus (1) comprise:
a first inlet port (25a);
a first outlet port (25b);
a first fluid conduit (25) configured for putting the first inlet port (25a) in fluid communication with the first outlet port (25b);
a second inlet port (26a);
a second outlet port (26b),
a second fluid conduit (26) configured for putting the second inlet port (26a) in fluid communication with the second outlet port (26b), the first fluid conduit (25) and the second fluid conduit (26) being fluidically separated from each other.

In a 5^{th} independent aspect, which may be combined with any other aspect or portion thereof described herein, a packaging for an extracorporeal blood treatment tubing set (2, 6, 7, 8) for an extracorporeal blood treatment apparatus (1) comprises a casing defining a hollow (sealed and possibly sterilized) openable housing arranged to accommodate the extracorporeal blood treatment tubing set (2, 6, 7, 8) comprising:
a blood treatment unit (2);
a fluid circuit (6) configured to be coupled with the blood treatment unit (2) and a warming/cooling bag (24) configured to be coupled with the fluid circuit (6), the warming/cooling bag (24) comprising:
   a first inlet port (25a);
   a first outlet port (25b);
   a first fluid conduit (25) configured for putting the first inlet port (25a) in fluid communication with the first outlet port (25b);
   a second inlet port (26a);
   a second outlet port (26b),
   a second fluid conduit (26) configured for putting the second inlet port (26a) in fluid communication with the second outlet port (26b), the first fluid conduit (25) and the second fluid conduit (26) being fluidically separated from each other and being empty.

Notably the first and second fluid conduits (25 and 26) define fluid flow paths that are isolated one from the other in the bag (24); no fluid in the first conduit (25) may flow and/or enter the second conduit (26) when inside the bag (24).

When in the packaging, the fluid conduits (25 and 25) are dry and do not contain any liquid.

In a 6^{th} aspect according to any one of the previous aspects, the first fluid conduit (25) comprises a first elongated fluid channel (25c) defining a flow direction for a corresponding fluid from the first inlet port (25a) to the first outlet port (25b).

In a 7^{th} aspect according to any one of the previous aspects, the first fluid conduit (25a) comprises:
a first inlet portion (25d) in direct communication with the first inlet port (25a);
a first outlet portion (25e) in direct communication with the first outlet port (25b); and,
a first intermediate portion (25f) connecting the first inlet portion (25d) to the first outlet portion (25e) and developing along a development direction, wherein the first intermediate portion (25f) has a substantially constant cross-sectional dimension.

In an 8^{th} aspect according to the previous aspect, the first inlet portion (25d) of the first fluid conduit (25) has a cross-sectional dimension increasing moving from the first inlet port (25a) to the first intermediate portion (25f).

In an 8^{th} bis aspect according to any one of the previous two aspects, the first outlet portion (25e) of the first fluid conduit (25) has a cross-sectional dimension decreasing moving from the first intermediate portion (25f) to the outlet port (25b).

In a 9^{th} aspect according to any one of the previous three aspects, the first intermediate portion (25f) of the first fluid conduit (25) includes:
two substantially parallel main tracts (25f′), optionally rectilinear;
a bend tract (25f″) connecting the main tracts (25f′), optionally arched, in particular circular.

In a 9^{th} bis aspect according to any one of the previous four aspects, the first intermediate portion (25f) of the first fluid conduit (25) has a U-shape or a C-shape configuration.

In a 10^{th} aspect according to any one of the previous aspects, the first inlet port (25a) and the first outlet port (25b) are placed on a same side (24a) of the fluid warming/cooling bag (24).

In an 11^{th} aspect according to any one of the previous aspects, the first fluid conduit (25) has only one first inlet port (25a) and has only one first outlet port (25b).

In a 12^{th} aspect according to any one of the previous aspects, the fluid warming/cooling bag (24) comprises a first inlet tube (28) having a constrained end (28a) connected to the first inlet port (25a) of the first fluid conduit (25) and a free end (28b), opposite to the constrained end (28a), provided with a connector (28c) for connection to a corresponding tube (7a, 7b, 8a), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood treatment apparatus (1).

In a 13^{th} aspect according to the previous aspect, the connector (28c) of the free end (28b) of the first inlet tube (28) of the first fluid conduit (25) is a Luer connector.

In a 14^{th} aspect according to any one of the previous two aspects, the connector (28c) of the free end (28b) of the first inlet tube (28) of the first fluid conduit (25) is a male connector, optionally a male Luer connector.

In a 15^{th} aspect according to any one of the previous three aspects, the fluid warming/cooling bag (24) comprises a first outlet tube (31) having a constrained end (31a) connected to the first outlet port (25b) of the first fluid conduit (25) and a free end (31b), opposite to the constrained end (31a), provided with a connector (31c) for connection to a corresponding tube (31), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood apparatus (1).

The fluid warming/cooling bag (24) is configured such that the fluid may enter/exit the first fluid conduit (265) exclusively from the free end (28b) of the first inlet tube (28) and from the free end (31b) of the first outlet tube (31).

In a 16^{th} aspect according to the previous aspect, the connector (31c) of the free end (31b) of the first outlet tube (31) of the first fluid conduit (25) is a Luer connector.

In a 17^{th} aspect according to any one of the previous two aspects, the connector (31c) of the free end (31b) of the first outlet tube (31) of the first fluid conduit (25) is a female connector, optionally a female Luer connector.

In an 18^{th} aspect according to any one of the previous aspects, the fluid warming/cooling bag (24) comprises a first marking to distinguish the first fluid conduit (25) with respect to the second fluid conduit (26).

In a 19^{th} aspect according to the previous aspect, the first marking is visual marking.

In a 20^{th} aspect according to any one of the previous two aspects, the first marking is a colour marking.

In a 21^{st} aspect according to any one of the previous three aspects, the first marking is a dimensional marking.

In a 22^{nd} aspect according to any one of the previous four aspects, the first marking is a shape marking.

In a 23^{rd} aspect according to any one of the previous five aspects when the 18^{th} aspect depends on any one of the aspects 12^{th} to 17^{th}, the first marking is placed on the first inlet tube (28) and/or on the first outlet tube (31), the first marking being an identifying colour.

In a 24^{th} aspect according to any one of the previous six aspects when the 18^{th} aspect depends on any one of the aspects 12^{th} to 17^{th}, the first marking is determined by the connector (28c) of the first inlet tube (28) and/or by the connector (31c) of the first outlet tube (31).

In a 25^{th} aspect according to any one of the previous seven aspects when the 18^{th} aspect depends on any one of the aspects 12^{th} to 17^{th}, the first marking is the length of the first inlet tube (28) and/or the length of the first outlet tube (31).

In a 26^{th} aspect according to any one of the previous aspects, the second fluid conduit (26) comprises a second elongated fluid channel (26c) defining a flow direction for a corresponding fluid from the second inlet port (26a) to the second outlet port (26b).

In a 27^{th} aspect according to any one of the previous aspects, the second fluid conduit (26) comprises:
a second inlet portion (26d) in direct communication with the second inlet port (26a);
a second outlet portion (26e) in direct communication with the second outlet port (26b); and,
a second intermediate portion (26f) connecting the second inlet portion (26d) to the second outlet portion (26e) and developing along a development direction, wherein the second intermediate portion (26f) has a substantially constant cross-sectional dimension.

In a 28^{th} aspect according to the previous aspect, the second inlet portion (26d) of the second fluid conduit (26) has a cross-sectional dimension increasing moving from the second inlet port (26a) to the second intermediate portion (26f).

In a 28^{th} bis aspect according to any one of the previous two aspects, the second outlet portion (26e) of the second fluid conduit (26) has a cross-sectional dimension decreasing moving from the second intermediate portion (26f) to the second outlet port (26b).

In a 29^{th} aspect according to any one of the previous three aspects, the cross-sectional dimension of the second fluid conduit (26) is different to the cross-sectional dimension of the first fluid conduit (25).

In a 30^{th} aspect according to any one of the previous four aspects, the second intermediate portion (26f) of the second fluid conduit (26) includes:
two substantially parallel main tracts (26f′), optionally rectilinear;
a bend tract (26f″) connecting the main tracts (26f′), optionally arched, in particular circular.

In a 30^{th} bis aspect according to any one of the previous five aspects, the first intermediate portion (26f) of the second fluid conduit (26) has a U-shape or a C-shape configuration.

In a 31^{st} aspect according to any one of the previous two aspects when depending on the aspect 9^{th} or any one previous aspect depending on the aspect 9^{th}, the two main tracts (25f′) and the bend track (25f″) of the first fluid conduit (25) are respectively substantially parallel to the two main tracts (26f′) and the bend track (26f″) of the second fluid conduit (26).

In a 32^{nd} aspect according to any one of the previous aspects when the 31^{st} aspect depends on the aspect 9^{th} or any one previous aspect depending on the aspect 9^{th}, the second fluid conduit (26) is placed among the two main tracts (25f′) and the bend track (25f″) of the first fluid conduit (25).

In a 33^{rd} aspect according to any one of the previous aspects, the second inlet port (26a) and the second outlet port (26b) are placed on a same side of the fluid warming/cooling bag (24).

In a 34^{th} aspect according to any one of the previous aspects, the first inlet port (25a) and the first outlet port (25b) of the first fluid conduit (25) and the second inlet port (26a) and the second outlet port (26b) of the second fluid conduit (26) are placed on a same side of the fluid warming/cooling bag (24).

In a 35^{th} aspect according to any one of the previous aspects, the second fluid conduit (26) has only one second inlet port (26a) and has only one second outlet port (26b).

In a 36^{th} aspect according to any one of the previous aspects, the fluid warming/cooling bag (24) comprises a second inlet tube (29) having a constrained end (29a) connected to the second inlet port (26a) of the second fluid conduit (26) and a free end (29b), opposite to the constrained end (29a), provided with a connector (29c) for connection to a corresponding tube (7a, 7b, 8a), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood apparatus (1).

In a 37^{th} aspect according to the previous aspect, the connector (29c) of the free end (29b) of the second inlet tube (??) of the second fluid conduit (26) is a Luer connector.

In a 38^{th} aspect according to any one of the previous two aspects, the connector (29c) of the free end (29b) of the second inlet tube (29) of the second fluid conduit (26) is a male connector, optionally a male Luer connector.

In a 39^{th} aspect according to any one of the previous three aspects, the fluid warming/cooling bag (24) comprises a second outlet tube (32) having a constrained end (32a) connected to the second outlet port (26b) of the second fluid conduit (26) and a free end (32b), opposite to the constrained end (32a), provided with a connector (32c) for connection to a corresponding tube (7a, 7b, 8a), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood apparatus (1).

The fluid warming/cooling bag (24) is configured such that the fluid may enter/exit the second fluid conduit (26) exclusively from the free end (29b) of the second inlet tube (29) and from the free end (32b) of the second outlet tube (32).

In a 40^{th} aspect according to the previous aspect, the connector (32c) of the free end (32b) of the second outlet tube (32) of the second fluid conduit (26) is a Luer connector.

In a 41^{st} aspect according to any one of the previous two aspects, the connector (32c) of the free end (32b) of the second outlet tube (32) of the second fluid conduit (26) is a female connector, optionally a female Luer connector.

In a 42^{nd} aspect according to any one of the previous aspects, fluid warming/cooling bag (24) comprises a second marking to distinguish the second fluid conduit (26) with respect to the first fluid conduit (25).

In a 43^{rd} aspect according to the previous aspect, the second marking is visual marking.

In a 44^{th} aspect according to any one of the previous two aspects, the second marking is a color marking.

In a 45^{th} aspect according to any one of the previous three aspects, the second marking is a dimensional marking.

In a 46^{th} aspect according to any one of the previous four aspects, the second marking is a shape marking.

In a 47^{th} aspect according to any one of the previous five aspects when the 42^{nd} aspect depends on any one of the aspects 36^{th} to 41^{st}, the second marking is placed on the first inlet tube (29) and/or on the first outlet tube (32), the second marking being an identifying colour.

In a 48^{th} aspect according to any one of the previous six aspects when the 42^{nd} aspect depends on any one of the aspects 36^{th} to 41^{st}, the second marking is the connector (29c) of the second inlet tube (29) and/or the connector (32c) of second outlet tube (32).

In a 49^{th} aspect according to any one of the previous seven aspects when the 42^{nd} aspect depends on any one of the aspects 36^{th} to 41^{st}, the second marking is determined by the length of the second inlet tube (29) and/or by the length of the second outlet tube (32).

In a 50^{th} aspect according to any one of the previous aspects, the fluid warming/cooling bag (24) comprises:
a third inlet port (27a);
a third outlet port (27b),
a third fluid conduit (27) configured for putting the third inlet port (27a) in fluid communication with the third outlet port (27b), the third fluid conduit (27) being fluidically separated from the first and the second fluid conduit (25, 26).

In a 51^{st} aspect according to the previous aspects, the third fluid conduit (27) comprises:
a third inlet portion (27d) in direct communication with the third inlet port (27a);
a third outlet portion (27e) in direct communication with the second outlet port (27b); and,
a third intermediate portion (27f) connecting the third inlet portion (27d) to the third outlet portion (27e) and developing along a development direction wherein the third intermediate portion (27f) has a substantially constant cross-sectional dimension.

In a 52^{nd} aspect according to the previous aspect, the third inlet portion (27d) of the third fluid conduit (27) has a cross-sectional dimension increasing moving from the third inlet port (27a) to the third intermediate portion (27f).

In a 52^{nd} bis aspect according to any one of the previous two aspects, the third outlet portion (27e) of the third fluid conduit (27) has a cross-sectional dimension decreasing moving from the third intermediate portion (27f) to the third outlet port (27b).

In a 53^{rd} aspect according to any one of the previous three aspects, the cross-sectional dimension of the third fluid conduit (27) is different to the cross-sectional dimension of at least one of the other fluid conduits (25, 27).

In a 54^{th} aspect according to any one of the previous four aspects, the cross-sectional dimension of the third fluid conduit (27) is different to the cross-sectional dimension of the first fluid conduit (25) and the cross-sectional dimension of the second fluid conduit (26).

In a 55^{th} aspect according to any one of the previous five aspects, the third intermediate portion (27f) of the third fluid conduit (27) includes:
two substantially parallel main tracts (27f′), optionally rectilinear;
a bend tract (27f″) connecting the main tracts (27f′), optionally arched, in particular circular.

In a 55^{th} bis aspect according to any one of the previous six aspects, the first intermediate portion (27f) of the third fluid conduit (27) has a U-shape or a C-shape configuration.

In a 56^{th} aspect according to any one of the previous two aspects when depending on the aspect 9^{th} or any one previous aspects depending on the aspect 9^{th}, the two main tracts (25f′) and the bend track (25f″) of the first fluid conduit (25) are respectively substantially parallel to the two main tracts (27f′) and the bend track (27f″) of the third fluid conduit (27).

In a 57^{th} aspect according to the previous two aspects when the aspect 55^{th} depends on the aspect 31^{st} or any one of the previous aspects depending on the aspect 31^{st}, the two main tracts (26f′) and the bend track (26f″) of the second fluid conduit (26) are respectively substantially parallel to the two main tracts (27f′) and the bend tract (27f″) of the third fluid conduit (27).

In a 58^{th} aspect according to any one of the previous three aspects when the 55^{th} aspect depends on the aspect 31^{st} or any one previous aspect depending on the aspect 31^{st}, the third fluid conduit (27) is placed among the two main tracts (26f′) and the bend track (26f″) of the second fluid conduit (26).

In a 59^{th} aspect according to any one of the previous aspects, the third inlet port (27a) and the third outlet port (27b) are placed on a same side of the fluid warming/cooling bag (24).

In a 60^{th} aspect according to any one of the previous aspects, the first inlet port (25a) and the first outlet port (25b) of the first fluid conduit (25), the second inlet port (26a) and the second outlet port (26b) of the second fluid conduit (26) and the third inlet port (27a) and the third outlet port (27b) of the third fluid conduit (27) are placed on a same side (24a) of the fluid warming/cooling bag (24).

In a 61^{st} aspect according to any one of the previous aspects, the third fluid conduit (27) has only one third inlet port (27a) and has only one third outlet port (27b).

In a 62^{nd} aspect according to any one of the previous aspects 50^{th} to 61^{st}, the fluid warming/cooling bag (24) comprises a third inlet tube (30) having a constrained end (30a) connected to the third inlet port (27a) of the third fluid conduit (27) and a free end (30b), opposite to the constrained end (27b), provided with a connector (30c) for connection to a corresponding tube (7a, 7b, 8a), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood apparatus (1).

In a 63^{rd} aspect according to the previous aspect, the connector (30c) of the free end (30b) of the third inlet tube (30) of the third fluid conduit (27) is a Luer connector.

In a 64^{th} aspect according to any one of the previous two aspects, the connector (30c) of the free end (30b) of the third inlet tube (30) of the third fluid conduit (27) is a male connector, optionally a male Luer connector.

In a 65^{th} aspect according to any one of the previous three aspects, the fluid warming/cooling bag (24) comprises a third outlet tube (33) having a constrained end (33a) connected to the third outlet port (27b) of the third fluid conduit (27) and a free end (33b), opposite to the constrained end (33a), provided with a connector (33c) for connection to a corresponding tube (7a, 7b, 8a), optionally a corresponding tube (7a, 7b, 8a) of the extracorporeal blood treatment tubing set (2, 6, 7, 8) or a corresponding tube (7a, 7b, 8a) of the fluid circuit (6) of the extracorporeal blood apparatus (1).

In a 66^{th} aspect according to the previous aspect, the connector (33c) of the free end (33b) of the third outlet tube (33) of the third fluid conduit (27) is a Luer connector.

In a 67^{th} aspect according to any one of the previous two aspects, the connector (33c) of the free end (33b) of the third outlet tube (33) of the third fluid conduit (27) is a female connector, optionally a female Luer connector.

In a 68^{th} aspect according to any one of the previous aspects, the fluid warming/cooling bag (24) comprises a third marking to allow the distinction of the third fluid conduit (27) with respect to the first fluid conduit (25) and/or the second fluid conduit (26).

In a 69^{th} aspect according to the previous aspect, the third marking is visual marking.

In a 70^{th} aspect according to any one of the previous two aspects, the third marking is a color marking.

In a 71^{st} aspect according to any one of the previous three aspects, the third marking is a dimensional marking.

In a 72^{nd} aspect according to any one of the previous four aspects, the second marking is a shape marking.

In a 73^{rd} aspect according to any one of the previous five aspects when the 68^{th} aspect depends on any one of the aspects 62^{nd} to 67^{th}, the third marking is placed on the third inlet tube (30) and/or on the third outlet tube (33), the third marking being an identifying colour.

In a 74^{th} aspect according to any one of the previous six aspects when the 68^{th} aspect depends on any one of the aspects 62^{nd} to 67^{th}, the third marking is the connector (30a) of the third inlet tube (30) and/or the connector (33c) of third outlet tube (33).

In a 75^{th} aspect according to any one of the previous seven aspects when the 68^{th} aspect depends on any one of the aspects 62^{nd} to 67^{th}, the third marking is the length of the third inlet tube (30) and/or the length of the third outlet tube (33).

In a 76^{th} aspect according to any one of the previous aspects, the fluid warming/cooling bag (24) is substantially flat.

When fluid flows inside the first fluid conduit (25) and/or the second fluid conduit (26), the fluid warming/cooling bag (24) assumes a more three dimensional shape as the fluid swells the first and second fluid conduits.

In a 77^{th} aspect according to any one of the previous aspects, the fluid warming/cooling bag (24) is flexible.

In a 78th aspect according to any one of the previous aspects, the fluid warming/cooling bag (24) is made of two sealed plastic films, the sealing defining (at least) the first and second fluid conduit (25, 26).

In a 79^{th} aspect according to any one of the previous aspects 50^{th} to 78^{th}, the fluid warming/cooling bag (24) is made of two sealed plastic films, the sealing defining the first (25), the second (26) and the third fluid conduit (27).

In an 80^{th} aspect according to any one of the previous aspects, the fluid warming/cooling bag (24) is made of medical grade plastic material, in specific, thermoplastic elastomer, for example Thermoplastic Polyurethane (TPU).

In an 81^{st} aspect according to any one of the previous aspects, the fluid warming/cooling bag (24) has a tab (34) protruding on a side (24b) opposite to the side (24a) wherein the first inlet port (25a, 26a, 27a) and the first outlet port (25b, 26b, 27b) of each corresponding fluid conduit (25, 26, 27) are placed.

In an 82^{nd} aspect according to the previous aspect, the tab (34) is positioned aside with respect to a median longitudinal axis (X) of the fluid warming/cooling bag (24), such that the blood warming/cooling bag (24) has an asymmetric shape.

In an 83^{rd} aspect according to the previous two aspects, the tab (34) has at least one through opening (34a), optionally two through openings (34a).

In an 84^{th} aspect according to any one of the previous aspects, the fluid warming/cooling bag (24) is disposable.

In an 84^{th} bis aspect according to any one of the previous aspects, the fluid warming/cooling bag (24) has a substantially rectangular shape with three substantially rectilinear sides, a first front side being interposed between two longer parallel second and third lateral sides, the first inlet port (25a) and the first outlet port (25b) being placed on the same first front side (24a) of the fluid warming/cooling bag (24).

In an 85^{th} aspect according to any one of the previous aspects, the fluid circuit (6) comprises an extracorporeal blood circuit (7) with a blood withdrawal line (7a) connected to an inlet port (3a) of the blood treatment unit (2) and a blood return line (7b) connected to an outlet port (3b) of the blood treatment unit (2), at least one fluid conduit (25, 26, 27) of the fluid warming/cooling bag (24) being in fluid communication with (or being configured to be in fluid communication with) the extracorporeal blood circuit (7) of the fluid circuit (6) to be supplied with blood (or blood infused with at least an infusion solution) withdrawn from a patient, treated, and to be reinfused into the patient.

In an 86^{th} aspect according to any one of the previous aspects, the fluid circuit (6) comprises a treatment fluid circuit (8) operatively connected to the extracorporeal blood circuit (7) and/or to the blood treatment unit (2), at least one fluid conduit (25, 26, 27) of the fluid warming/cooling bag (24) being in fluid communication with or being configured to be in fluid communication with the treatment fluid circuit (8) of the fluid circuit (6) to be supplied with a treatment fluid or an infusion solution.

In an 87^{th} aspect according to any one of the previous aspects, the fluid circuit (6) comprises:
an extracorporeal blood circuit (7) with a blood withdrawal line (7a) connected to an inlet port (3a) of the blood treatment unit (2) and a blood return line (7b) connected to an outlet port (3b) of the blood treatment unit (2), one fluid conduit (25, 26, 27) of the fluid warming/cooling bag (24) being in fluid communication with or being configured to be in fluid communication with the extracorporeal blood circuit (7) of the fluid circuit (6) to be supplied with blood or blood infused with at least an infusion solution;
a treatment fluid circuit (8) operatively connected to the extracorporeal blood circuit (7) and/or to the blood treatment unit (2), the other fluid conduit (25, 26, 27) of the fluid warming/cooling bag (24) being in fluid communication with or being configured to be in fluid communication with the treatment fluid circuit (8) of the fluid circuit (6) to be supplied with a treatment fluid or an infusion solution.

In an 88^{th} aspect according to any one of the previous aspects, at least one fluid conduit of the fluid warming/cooling bag (24) is directly in fluid communication with or is configured to be directly in fluid communication with at least one source of an infusion solution. The source may be a bag filled with the infusion solution.

In an 89^{th} aspect according to any one of the previous aspects, the at least one fluid conduit (25, 26, 27) of the fluid warming/cooling bag (24) is in fluid communication with or is configured to be in fluid communication with at least one source of an infusion solution through at least a portion of the fluid circuit (6).

In an 90^{th} aspect according to the aspect 55^{th} or any one of the previous aspects from 56^{th} to 89^{th} when depending on the aspects 55^{th}, the fluid circuit (6) comprises:
an extracorporeal blood circuit (7) with a blood withdrawal line (7a) connected to an inlet port (3a) of the blood treatment unit (2) and a blood return line (7b) connected to an outlet port (7b) of the blood treatment unit (2), one fluid conduit (25, 26, 27) of the fluid warming/cooling bag (24) being in fluid communication with or being configured to be in fluid communication with the extracorporeal blood circuit (7) of the fluid circuit (2) to be supplied with blood or blood infused with at least an infusion solution;
a treatment fluid circuit (8) operatively connected to the extracorporeal blood circuit (7) and/or to the blood treatment unit (2), one fluid conduit (25, 26, 27) of the fluid warming/cooling bag (24) being in fluid communication with or being configured to be in fluid communication with the treatment fluid circuit (8) of the fluid circuit (6) to be supplied with a treatment fluid or an infusion solution;
a supply line (12, 13, 14a, 14b) of an infusion solution operatively connected to the extracorporeal blood circuit (7) and/or to the treatment fluid circuit (8), one fluid conduit (25, 26, 27) of the fluid warming/cooling bag (24) being in fluid communication with or being configured to be in fluid communication with the supply line (12, 13, 14a, 14b) of the infusion solution to be supplied with such infusion solution.

In a 91^{st} aspect according to any one of the previous aspects, the temperature control device (15) is a warming device configured to heat the warming/cooling bag (24), the heat exchange region (18) of the temperature control device (15) being a heating region.

In a 92^{nd} aspect according to any one of the previous aspects, at least one deaeration/filtration device is operatively connected or is configured to be connected to at least one of the outlet ports (25b, 26b, 27b) of the fluid conduits (25, 26, 27) of the fluid warming/cooling bag (24), optionally the deaeration/filtration device is operatively connected or is configured to be connected to each outlet port (25b, 26b, 27b) of the fluid conduits (25, 26, 27) of the fluid warming/cooling bag (24).

In a 93^{rd} aspect according to the previous aspect, the deaeration/filtration device is included into or forms a part of the fluid circuit (6).

In a 94^{th} aspect according to any one of the previous two aspects, the deaeration/filtration device is included into or forms a part of the extracorporeal blood treatment tubing set (2, 6, 7, 8).

In a 95^{th} aspect according to any one of the previous aspects, the extracorporeal blood treatment tubing set (2, 6, 7, 8) is a disposable set.

In a 96^{th} aspect according to any one of the previous aspects, the temperature control device (15) is configured to receive the warming/cooling bag (24) in a respective seat (or receptacle) substantially counter-shaped to the warming/cooling bag (24).

In a 97^{th} aspect according to any one of the previous aspects, the temperature control device (15) comprises at least one (and particularly two) heating element/s (or first temperature exchange element (22) and second temperature exchange element (23) active on the warming/cooling bag (24) when coupled to the temperature control device (15).

In a 98^{th} aspect according to any one of the previous aspects, the controller (35) is configured to control one or more of the following parameters:
a fluid flow rate of a fluid in the first fluid conduit (25);
a fluid flow rate of a fluid in the second fluid conduit (26);
a heating (e.g., a heating power or a heating temperature) of a first temperature exchange element (22) of the temperature control device (15);
a heating (e.g., a heating power or a heating temperature) of a second temperature exchange element (23) of the temperature control device (15).

In a 99^{th} aspect according to the previous aspect, the controller (35) is configured to control one or more of the parameters based on a signal from a temperature sensor placed on the fluid circuit (6), particularly on the extracorporeal blood circuit (7), such as on the blood return line (7b) downstream the fluid warming/cooling bag (24).

### BRIEF DESCRIPTION OF THE FIGURES

FIG. 1 is a schematic view of an extracorporeal blood treatment apparatus provided with a temperature control device according to present disclosure;

FIG. 2 is a schematic perspective view of the temperature control device included into the extracorporeal blood treatment apparatus of FIG. 1;

FIG. 3 is a schematic perspective view of the temperature control device FIG. 2, shown in an open configuration;

FIG. 4 is a schematic plan view of an exemplificative fluid warming/cooling bag to be used into the temperature control device of FIGS. 1 to 3 in order to regulate the temperature of at least on medical fluid.

### DETAILED DESCRIPTION

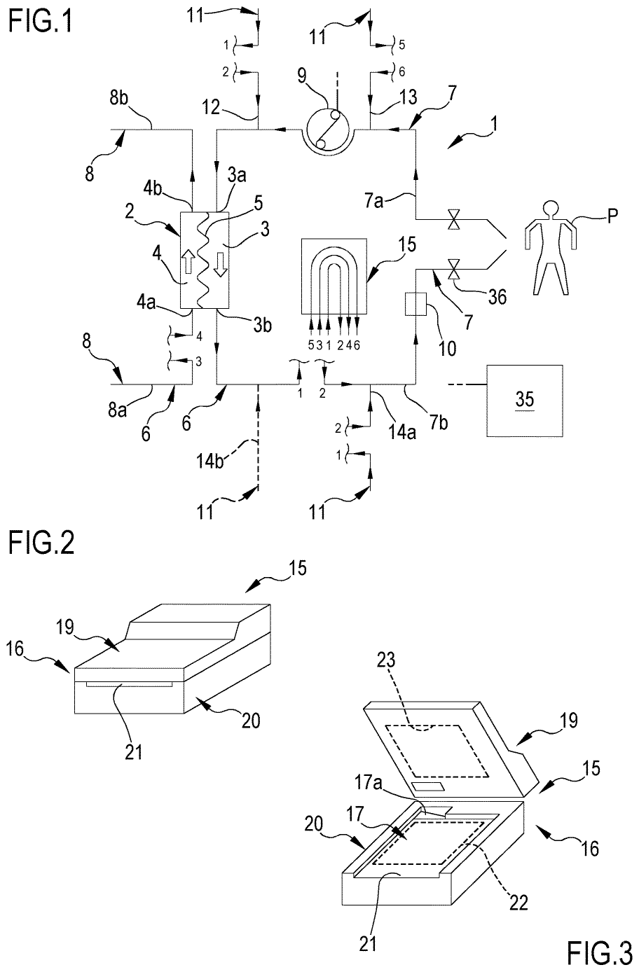

With reference to the appended drawings, FIG. 1 shows a schematic view of an extracorporeal blood treatment apparatus 1.

The apparatus 1 comprises a blood treatment unit 2, for example a hemofilter, a hemodiafilter, a plasma separator, a dialysis filter, an absorber, a hemoperfusor, a gas exchanger, such as a carbon dioxide remover, or other unit suitable for processing the blood taken from a patient P.

The blood treatment unit 2 may have a first compartment or blood chamber 3 and a second compartment or fluid chamber 4 separated from one another by a semipermeable membrane 5.

The apparatus 1 also comprises a fluid circuit 6 coupled to the blood treatment unit 2. In particular, the fluid circuit 6 of the apparatus 1 includes an extracorporeal blood circuit 7 with a blood withdrawal line 7a connected to an inlet port 3a of the first compartment or blood chamber 3 of the blood treatment unit 2 and a blood return line 7b connected to an outlet port 3b of the blood treatment unit 2 and a treatment fluid circuit 8 operatively connected to the extracorporeal blood circuit 7 and/or to the blood treatment unit 2.

Considering FIG. 1, the blood withdrawal line 7a of the extracorporeal blood circuit 7 is configured, in an operative condition, of connection to the patient P, to withdraw blood from the patient P via a vascular access device inserted, for example in a fistula on the patient P and the blood return line 7b is configured to receive treated blood from the blood treatment unit 2 and to return the treated blood, e.g. to a further vascular access also connected to the fistula of the patient P. Note that various configurations for the vascular access device may be envisaged: for example, typical access devices include a needle or catheter inserted into a vascular access which may be a fistula, a graft or a central (e.g. jugular vein) or peripheral vein (femoral vein) and so on.

The extracorporeal blood circuit 7 and the treatment unit 2 are usually disposable parts which are loaded onto a frame of a blood treatment apparatus, not shown. The blood treatment apparatus cooperates with the extracorporeal blood circuit 7 and the treatment unit 2 to perform the extracorporeal blood treatment, such as the dialysis treatment on the patient P.

As shown in FIG. 1, the apparatus 1 comprises at least a fluid pump 9, in the present example a blood pump, which is part of the machine and works on a tube pump section of the blood withdrawal line 7a, to cause movement of the blood removed from the patient P from a first end of the blood withdrawal line 7a connected to the patient P to the blood chamber 3 of the blood treatment unit 2. The fluid pump 9 is, for example, a peristaltic pump, as shown in FIG. 1, which acts on a respective pump section of the blood withdrawal line 7a of the extracorporeal blood circuit 7.

It should be noted that for the purposes of the present disclosure and the appended claims, the terms “upstream” and “downstream” may be used with reference to the relative positions taken by components belonging to or operating on the fluid circuit 6 and, in particular with reference to the extracorporeal blood circuit and/or the treatment fluid circuit 8. These terms are to be understood with reference to a fluid flow direction. Taking as an example the extracorporeal blood circuit 7 of the fluid circuit 6, the above mentioned terms are to be understood with reference to the blood flow direction from the first end of the blood withdrawal line 7a connected to the patient P towards the blood chamber 3 of the blood treatment unit 2 and then from the blood chamber 3 of such unit 2 towards a second end of the blood return line 7b connected to the vascular access of the patient P.

The apparatus further comprises a controller 35 to control the fluid pump 9 and other pumps of the apparatus. The controller 35 is also operatively connected to sensors (like flow sensors and/or pressure sensors) on the blood circuit and/or fluid circuit and/or infusion circuit. The controller 35 is also operatively connected to clamps and valves, like a return clamp 36 to close the blood circuit and interrupt blood circulation in case of an alarm requiring treatment interruption. The control unit 10 is also connected to the user interface, not shown, for instance a graphic user interface, which receives operator's inputs and displays the apparatus outputs. For instance, the graphic user interface may include a touch screen, a display screen and hard keys for entering user's inputs or a combination thereof. During extracorporeal blood treatment, the controller 35 is configured to control at least the pumps to make sure that a prefixed patient net fluid removal is achieved in the course of a treatment time, as required by a prescription provided to controller 35, e.g. via the user interface.

The controller 35 may comprise a digital processor (CPU) with memory (or memories), an analogical type circuit, or a combination of one or more digital processing units with one or more analogical processing circuits. In the present description and in the aspects it is indicated that the controller 35 is “configured” or “programmed” to execute steps: this may be achieved in practice by any means which allow configuring or programming the controller 35. For instance, in case of a controller 35 comprising one or more CPUs, one or more programs are stored in an appropriate memory: the program or programs containing instructions which, when executed by the controller 35, cause the controller 35 to execute the steps described and/or claimed in connection with the controller 35. Alternatively, if the controller 35 is of an analogical type, then the circuitry of the controller 35 is designed to include circuitry configured, in use, to process electric signals such as to execute the controller 35 steps herein disclosed.

The apparatus 1 may further comprise an air trapping device 10 operating on the blood return line 7b (the air trapping device 10 may be a venous deaeration chamber) of the extracorporeal blood circuit 7. The air trapping device 10 is placed online in the blood return line 7b of the extracorporeal blood circuit 7.

A first section of the blood return line 7b of the extracorporeal blood circuit 7 puts in fluid communication the outlet port 3b of the blood chamber 3 with the air trapping device 10 and a second section of the blood return line 7b puts in fluid communication the air trapping device 10 with the patient P. The blood coming from the blood chamber 3 of the treatment device 2 enters and exits the air trapping device 10 before reaching the patient P.

Always with reference to FIG. 1, the treatment fluid circuit 8 of the fluid circuit 6 of the extracorporeal blood treatment apparatus 1 further comprises a fluid evacuation line 8b connected with an outlet port 4b of the fluid chamber 4 of the blood treatment unit 2 such as to receive the filtered waste fluid through the semipermeable membrane 5. The fluid evacuation line 8b receives the spent dialysis fluid coming from the fluid chamber 4 of the blood treatment unit 2, for example, comprising used dialysis liquid and/or liquid ultra-filtered through the membrane 5. The fluid evacuation line 8b leads to a receiving element, not shown, for example having a collection bag or a drainage pipe for the waste fluid. One or more dialysate pumps, not shown, may operate on the fluid evacuation line 8b.

In the example of FIG. 1, a dialysis line 8a of the treatment fluid circuit 8 is also present for supplying a fresh treatment fluid into the inlet port 4a of the fluid chamber 4 of the blood treatment unit 2. Even if the presence of the dialysis line 8a is the usual choice, it has to be noted that it is not strictly necessary since, in the absence of the dialysis line 8a, the apparatus 1 is still able to perform treatments such as ultrafiltration, hemofiltration or plasma-filtration. In case the dialysis line 8a is present, a fluid flow intercept device may be used, not shown, to selectively allow or inhibit fluid passage through the dialysis line 8a of the treatment fluid circuit 8, depending on whether or not a purification by diffusive effect is to be performed inside the blood treatment unit 2.

The dialysis line 8a, if present, is typically equipped with a dialysis pump and is able to receive a fresh fluid from a module, not shown, for example a bag or on-line preparation section of dialysis fluid, and to send such a fluid to the inlet port 4a of the fluid chamber 4 of the blood treatment unit 2.

The fluid evacuation line 8b, the dialysis line 8a, and the fluid chamber 4 are part of a treatment fluid circuit 8 of the fluid circuit 6.

The apparatus 1 as shown in FIG. 1 may also comprise an infusion circuit 11 comprising one or more infusion lines 12, 13, 14a, 14b of a replacement fluid, also named replacement lines.

According to the embodiment of FIG. 1, a pre-infusion line 12 is connected to the blood withdrawal line 7a of the blood extracorporeal circuit 7 between the blood pump 9 and the inlet port 3a of the blood chamber 3 of the blood treatment unit 2. A pre-pump infusion line 13 may be connected to the blood withdrawal line 7a of the blood extracorporeal circuit 7 upstream of the blood pump 9, between this latter and the vascular access device inserted in the fistula on the patient P.

At least one post-replacement line 14a, 14b may be connected to the blood return line 7b of the extracorporeal blood circuit 7 for performing HF or HDF treatments. Generally one or two post-replacement lines 14a, 14b are used connected upstream of or to the air trapping device 10. FIG. 1 shows that the post-replacement line 14a, 14b comprises a first 14a and a second branch 14b. Each of the pre-and/or post-replacement lines 12, 13, 14a, 14b is provided with a respective pump, not shown. The pre-and/or post-replacement lines 12, 13, 14a, 14b may be supplied by fluid coming from bags or directly by replacement fluid prepared on-line. Each of the pre-and/or post-replacement lines 12, 13, 14a, 14b may be part of the treatment fluid circuit 8. The specific configuration of the pre-and post-replacement lines 12, 13, 14a, 14b may of course differ from those shown in FIG. 1.

The apparatus 1 comprises a fluid temperature control device 15 operatively associated with the fluid circuit 6 to regulate the temperature of at least one medical fluid flowing through the fluid circuit 6. The fluid temperature control device 15 may be part of the extracorporeal blood treatment apparatus 1 or an external device capable of exchanging data with the controller 35 of the apparatus in order to properly control the heating of the fluids.

With reference to the appended FIGS. 1 and 2, the temperature control device 15 comprises a casing 16 delimiting a receptacle 17 where a heat exchange region 18 is active. The receptacle is substantially counter-shaped to a corresponding fluid warming/cooling bag 24. The casing 16 comprises an upper part 19 and a lower part 20 properly linked and in an example movable between a working configuration (shown in FIG. 2) and a maintenance configuration (shown in FIG. 3). The upper part 19 and lower part 20 of FIGS. 2 and 3 are hinged to move between the mentioned configurations. When the casing 16 is in the working configuration of FIG. 2, the upper part 19 and the lower part 20 are juxtaposed and delimit inside the casing 16 the corresponding receptacle 17 which opens outside through a slot 21, optionally having a height comprised between 1.3 mm and 3.2 mm.

When the casing 16 is in the maintenance configuration of FIG. 3, the upper part 19 is rotated and raised with respect to the lower part 20 to open the receptacle 17 and to allow maintenance operations and/or cleaning.

An upper face of the lower part 20 of the casing 16 may be hollow, presenting a first (e.g. flat) surface or plate provided with a first temperature exchange element 22 placed underneath the first surface or plate. A lower face of the upper part 19 has a second (e.g., flat) surface or plate that may be provided with a second temperature exchange element 23 placed underneath the second surface or plate. The first surface and the second surface are opposite (and in the shown example parallel) delimiting respectively the lower part and the upper part of the receptacle 17.

The first and the second temperature exchange elements 22, 23 of the temperature control device 15 may be arranged to bring about either a heating or a cooling action inside the receptacle 17.

In particular, the first and the second temperature exchange elements 22, 23 of the temperature control device 15 are heating elements that heat the receptacle 17, being the temperature control device 15 a warmer. The heating elements are configured to be electrical connected to electrical resistors powered by a power source and controlled by an electronic control unit, e.g., controller 35. Either (or both) the first or the second temperature exchange elements 22, 23 can be heated as a function of the heating power to be transferred.

The apparatus 1 also comprises a fluid warming/cooling bag 24, advantageously disposable, configured to be coupled to the fluid circuit 6 to receive at least one medical fluid. The fluid warming/cooling bag 24 is also configured to be accommodated into the receptacle 17 of the fluid temperature control device 15 in order to be subjected to at least one temperature control operation, optionally a periodic or continuous temperature control operations.

Alternatively, the fluid warming/cooling bag 24 may also not be part of the apparatus 1 and being nevertheless coupled to the fluid circuit 6 and accommodated to the receptacle 17 of the fluid temperature control device 15.

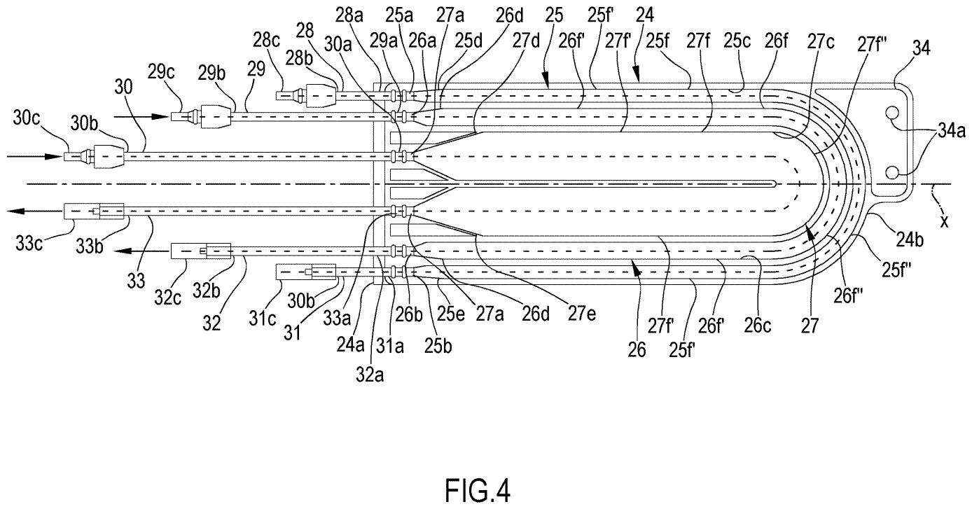

With reference to FIG. 4, the fluid warming/cooling bag 24 comprises a first inlet port 25a, a first outlet port 25b and a first fluid conduit 25 configured for putting the first inlet port 25a in fluid communication with the first outlet port 25a. The fluid warming/cooling bag 24 also comprises a second inlet port 26a, a second outlet port 26b and a second fluid conduit 26 configured for putting the second inlet port 26a in fluid communication with the second outlet port 26a. The first fluid conduit 25 and the second fluid conduit 26 are fluidically and hermetically separated from each other.

Considering the embodiment shown in FIG. 4, the fluid warming/cooling bag 24 further comprises a third inlet port 27a, a third outlet port 27b and a third fluid conduit 27 configured for putting the third inlet port 27a in fluid communication with the third outlet port 27a. Also the third fluid conduit 27 is fluidically and hermetically separated from the first and the second conduits 25, 26 are fluidically and hermetically separated from each other, whereby a medical fluid flowing through a specific fluid conduit 25, 26, 27 of the fluid warming/cooling bag 24 cannot leak into another fluid conduit 25, 26, 27.

Usually the the fluid warming/cooling bag 24 has only two fluid conduits 25, 26 but, in some cases, it may be designed to have more than two fluid conduits like the embodiment shown in FIG. 4 wherein the fluid warming/cooling bag 24 has three fluid conduits 25, 26, 27.

Each fluid conduit 25, 26, 27 has only one corresponding inlet port 25a, 26a 27a and has only one corresponding outlet port 25b, 26b, 27b.

It has to be noted that the number of the fluid conduits of the fluid warming/cooling bag 24 may vary according to the need, being however limited by dimensional and structural limits of the fluid warming/cooling bag 24 itself.

When the fluid warming/cooling bag 24 is accommodated in the receptacle 17, the fluid temperature control device 15 is configured to determine a heat exchange with at least one fluid inside one of the fluid conduits 25, 26, 27 of the warming/cooling bag 24 at the heat exchange region 18. In particular, when the fluid warming/cooling bag 24 is accommodated in the receptacle 17, the fluid temperature control device 15 is configured to determine a heat exchange with all fluids inside the fluid conduits 25, 26, 27 of the warming/cooling bag 24.

By further detailing the structure of the fluid warming/cooling bag 24 of FIG. 4, each fluid conduit 25, 26, 27 comprises an elongated fluid channel 25c, 26c, 27c defining a flow direction for a corresponding fluid from the first inlet port 25a, 26a, 27a to the first outlet port 25b, 26b, 27b.

More in detail, each fluid conduit 25, 26, 27 comprises an inlet portion 25d, 26d, 27d in direct communication with the corresponding inlet port 25a, 26a 27a, a corresponding outlet portion 25e, 26e, 27e in direct communication with the corresponding outlet port 25b, 26b, 27b and a corresponding intermediate portion 25f, 26f, 27f connecting the corresponding inlet portion 25d, 26d, 27d to the corresponding outlet portion 25e, 26e, 27e and developing along a development direction.

Each fluid conduit 25, 26, 27 has a respective intermediate portion 25f, 26f, 27f with a substantially constant cross-sectional dimension, an inlet portion 25d, 26d, 27d with a cross-sectional dimension increasing moving from the corresponding inlet port 25a, 26a, 27a to the corresponding intermediate portion 25f, 26f, 27f, and a corresponding outlet portion 25e, 26e, 27e with a cross-sectional dimension decreasing moving from the corresponding intermediate portion 25f, 26f, 27f towards the corresponding outlet port 25b, 26b, 27b.

Moreover, the intermediate portion 25f, 26f, 27f of each fluid conduit 25, 26, 27 includes two substantially parallel main tracts 25f′, 26f′, 27f′, optionally rectilinear and a corresponding bend tract 25f″, 26f″, 27f″ connecting the main tracts 25f′, 26f′, 27f′, optionally arched, in particular circular.

As visible in FIG. 4, the parallel main tracts 25f′, 26f′, 27f′ and the corresponding bend tract 25f″, 26f″, 27f″ of the corresponding intermediate portion 25f, 26f, 27f of each fluid conduit 25, 26, 27 of the fluid warming/cooling bag 24 define corresponding U-shape or a C-shape configurations.

Advantageously, the inlet port 25a, 26a 27a and the corresponding outlet port 25b, 26b, 27b of each fluid conduit 25, 26, 27 are placed on a same side 24a of the fluid warming/cooling bag 24.

Looking at the embodiment shown in FIG. 4, it is possible to see that the fluid warming/cooling bag 24 comprises an inlet tube 28, 29, 30 having a constrained (non removable) end 28a, 29a, 30a connected to the corresponding inlet port 25a, 26a 27a of the corresponding fluid conduit 25, 26, 27 and a free end 28b, 29b, 30b, opposite to the corresponding constrained end 28a, 29a, 30a, provided with a connector 28c, 29c, 30c for connection to a corresponding tube 7a, 7b, 8a, optionally a corresponding tube 7a, 7b, 8a of an extracorporeal blood treatment tubing set 2, 6, 7, 8 or a corresponding tube 7a, 7b, 8a of the fluid circuit 6 of the extracorporeal blood apparatus 1.

Moreover, the fluid warming/cooling bag 24 comprises an outlet tube 31, 32, 33 having a constrained (non removable) end 31a, 32a, 33a connected to the corresponding outlet port 25b, 26b, 27b of the corresponding fluid conduit 25, 26, 27 and a free end 31b, 32b, 33b, opposite to the constrained end 31a, 32a, 33a, provided with a connector 31c, 32c, 33c for connection to a corresponding tube 7a, 7b, 8a, optionally a corresponding tube 7a, 7b, 8a of an extracorporeal blood treatment tubing set 2, 6, 7, 8 or a corresponding tube 7a, 7b, 8a of the fluid circuit 6 of the extracorporeal blood apparatus 1.

The connector 28c, 29c, 30c of the free end 28b, 29b, 30b of the corresponding inlet tube 28, 29, 30 of each fluid conduit 25, 26, 27 is a male connector, optionally a male Luer connector. The connector 31c, 32c, 33c of the free end 31b, 32b, 33b of the corresponding outlet tube 31, 32, 33 of each fluid conduit 25, 26, 27 is a female connector, optionally a female Luer connector.

In order to facilitate the immediate and unequivocal identification of each fluid conduit 25, 26, 27 with respect to the others, allowing also a clear and easy identification of the medical fluid flowing through such fluid conduits 25, 26, 27, the fluid warming/cooling bag 24 comprises corresponding markings, optionally visual markings, associated with the fluid conduits 25, 26, 27.

In particular, according to an embodiment of the fluid warming/cooling bag 24 the marking of each fluid conduit 25, 26, 27 is a colour marking. The colour marking may be defined by a coloured external pigmentation of the corresponding fluid conduit 25, 26, 27 and/or the corresponding inlet tube 28, 29, 30 and/or the corresponding outlet tube 31, 32, 33, or a portion of them. Alternatively the color marking may be defined by a colored label/tag, ring, sign properly attached or engaged to the corresponding fluid conduit 25, 26, 27 and/or the corresponding inlet tube 28, 29, 30 and/or the corresponding outlet tube 31, 32, 33.

Naturally, any other type of colour markings may be used at place of the ones previously cited to the extent that such colour markings allow a sudden identification of each fluid conduit 25, 26, 27.

According to another embodiment of the fluid warming/cooling bag 24 the marking of each fluid conduit 25, 26, 27 is a dimensional marking. The dimensional marking may be defined by different sizes of the corresponding fluid conduit 25, 26, 27 and/or the corresponding inlet tube 28, 29, 30 and/or the corresponding outlet tube 31, 32, 33, or a portion of them. In particular, the dimensional marking may be defined by a specific length and/or a specific width of the inlet and/or outlet tubes.

According to further embodiment of the fluid warming/cooling bag 24, the first marking is a shape marking. The shape marking may be defined by a specific profile of corresponding fluid conduit 25, 26, 27, in particular a specific profile of the corresponding bend tract 25f″, 26f″, 27f″. In addition or alternatively, it is possible to provide fins or flaps or any other structural element attached or engaged to the corresponding inlet tube 28, 29, 30 and/or the outlet tube 31, 32, 33, the shape of which allows the immediate identification of the corresponding fluid conduit 25, 26, 27.

According to another embodiment of the fluid warming/cooling bag 24, the shape marking may be also defined by the external appearances of the connector of the corresponding inlet tube 28, 29, 30 and/or outlet tube 31, 32, 33.

In order to be housed into the receptacle 17 of the temperature control device 15 with the inlet tubes 28, 29, 30 and the outlet tubes 31, 32, 33 of the corresponding fluid conduits 25, 26, 27 developing across the slot 21 of the casing 16, the fluid warming/cooling bag 24 is substantially flat and presents an overall thickness between 1.5 mm to 3 mm, being adapted to be inserted through the slot 21.

Advantageously, the fluid warming/cooling bag 24 is soft and flexible, is made of two sealed plastic films. The sealing of the plastic films defines the fluid conduits 25, 26, 27.

The fluid warming/cooling bag 24 is made of thermoplastic elastomer, e.g., Thermoplastic Polyurethane (TPU) so that to have a good thermal stability and flexibility along with stable and elastic properties at the body temperature without the need of adding any processing agent.

Looking at FIG. 4, the fluid warming/cooling bag 24 has a tab 34 protruding on a side 24b opposite to the side 24a where inlet ports 25a, 26a, 27a and the outlet ports 25b, 26b, 27b are placed. The tab 34 has two through openings 34a and is positioned aside with respect to a median longitudinal axis X of the fluid warming/cooling bag 24, such that the blood warming/cooling bag 24 has an asymmetric shape. The tab 24 is arranged to accommodate a corresponding recess 17a of the receptacle 17 of the temperature control device 15.

Irrespective of the number of the fluid conduits 25, 26, 27 of the fluid warming/cooling bag 24, each fluid conduit 25, 26, 27 may be connected to the fluid circuit 6 of the extracorporeal blood circuit and/or external fluid circuits outside the fluid circuit 6, like for instance, an external infusion circuit, to receive a specific medical fluid which temperature needs to be controlled and regulated.

In particular, whether it is needed to control the temperature of the patient's blood at least one of the fluid conduits 25, 26, 27 of the fluid warming/cooling bag 24 is connected to the extracorporeal blood circuit 7. When at least one fluid conduit 25, 26, 27 is connected to the extracorporeal blood circuit 7 upstream the blood treatment unit, i.e. at the blood withdrawal line 7a, the temperature regulation carried out by the temperature control device 15, is effected on the patient's blood not already treated. When at least one fluid conduit 25, 26, 27 is connected to the extracorporeal blood circuit 7 downstream the blood treatment unit 2, i.e. at the blood return line 7b, the temperature regulation carried out by the temperature control device 15 is effected on the blood treated before returning to the patient P.

In the event that the fluid warming/cooling bag 24 has two or more fluid conduits 25, 26, 27, it is also possible to provide for a double blood temperature control/regulation in series, i.e. leading firstly the patient's blood flowing through the blood withdrawal line 7a across a corresponding fluid conduit 25, 26, 27, and leading secondly the treated blood flowing through the blood return line 7b across another corresponding fluid conduit 25, 26, 27. In this manner the temperature of both systemic blood and treated blood is controlled/regulated simultaneously.

In order to allow the connection between the fluid conduits 25, 26, 27 to the extracorporeal blood circuit 7, it is sufficient to connect the corresponding connectors 28c, 29c, 30c, 31c, 32c, 33c of the corresponding inlet tubes 28, 29, 30 and outlet tube 31, 32, 33 to the specific blood lines 7a, 7b.

Whether it is needed to control the temperature of the treatment fluid supplied to the blood treatment unit 2 through the dialysis line 8a of the treatment fluid circuit 8, at least one of the fluid conduits 25, 26, 27 of the fluid warming/cooling bag 24 has to be connected to the dialysis line 8a upstream the blood treatment unit 2.

Supposing a fluid warming/cooling bag 24 provided with only two fluid conduits 25, 26, 27, a fluid conduit 25, 26, 27 is connected to the blood withdrawal line 7a or the blood return line 7b of the extracorporeal blood circuit 7, as previously explained and the other fluid conduit 25, 26, 27, whereas the other fluid conduit is connected to the dialysis line 8a of the treatment fluid circuit 8.

Of course, in a different embodiment, one fluid conduit may be connected to an infusion line (e.g., a post infusion line), while the other fluid conduit is connected to the dialysis line 8a to warm two different fluids that enter into contact with blood thereby warming the blood.

Assuming instead that the fluid warming/cooling bag 24 has three fluid conduits 25, 26, 27 as in the embodiment of FIG. 4, nothing prevents one fluid conduit 25, 26, 27 being connected to the blood extracorporeal circuit 7, e.g., to the blood return line 7b, whereas the remaining two fluid conduits 25, 26, 27 are connected to the dialysis line 8a of the treatment fluid circuit 8 and to an infusion line.

Also in this case, in order to allow the connection between the fluid conduits 25, 26, 27 to the lines 7a, 7b of the extracorporeal blood circuit 7 or the dialysis line 8a of the treatment fluid circuit 8, it is sufficient to connect the corresponding connectors 28c, 29c, 30c, 31c, 32c, 33c of the corresponding inlet tubes 28, 29, 30 and outlet tube 31, 32, 33 to the specific lines 7a, 7b, 8a of the fluid circuit 6 of the apparatus 1.

Should at least one replacement solution or any other fluid (from now on called additional fluid) different to blood or the treatment fluid supplied to the fluid circuit 6 or even directly supplied to the patient P require to be regulated in temperature before reaching its destination, it is possible to allocate at least one fluid conduit 25, 26, 27 to such a fluid.

Supposing a fluid warming/cooling bag 24 provided with only two fluid conduits 25, 26, 27, the possible combination of correspondence between the fluid conduits 25, 26, 27 and the fluids which temperature needs to be controlled, are:
1—one fluid conduit 25, 26, 27 for one additional fluid, which may be an replacement solution before entering into a corresponding circuit 6, 7, 8 or one additional fluid directly supplied to the patient P and one fluid conduit 25, 26, 27 for blood;
2—one fluid conduit 25, 26, 27 for one additional fluid, which may be an replacement solution before entering into a corresponding circuit 6, 7, 8 or one additional fluid directly supplied to the patient P and one fluid conduit 25, 26, 27 for treatment fluid;
3—one fluid conduit 25, 26, 27 for one additional fluid, which may be an replacement solution before entering into a corresponding circuit 6, 7, 8 or one additional fluid directly supplied to the patient P and one fluid conduit 25, 26, 27 for another additional fluid.

Supposing a fluid warming/cooling bag 24 provided with three fluid conduits 25, 26, 27, as shown in FIG. 4, the possible combination of correspondence between the fluid conduits 25, 26, 27 and the fluids which temperature needs to be controlled, are:
1—one fluid conduit 25, 26, 27 for one additional fluid, which may be an replacement solution before entering into a corresponding circuit 6, 7, 8 or an additional fluid directly supplied to the patient P, one fluid conduit 25, 26, 27 for blood and one fluid conduit 25, 26, 27 for treatment fluid;
2—one fluid conduit 25, 26, 27 for one additional fluid, which may be an replacement solution before entering into a corresponding circuit 6, 7, 8 or one additional fluid directly supplied to the patient P and two fluid conduits 25, 26, 27 for blood, in particular systemic and treated;
3—two fluid conduits 25, 26, 27 for additional fluids, which may be replacement solutions before entering into a corresponding circuit 6, 7, 8 or additional fluids directly supplied to the patient P and one fluid conduit 25, 26, 27 for blood;
4—two fluid conduits 25, 26, 27 for additional fluids, which may be replacement solutions before entering into a corresponding circuit 6, 7, 8 or additional fluids directly supplied to the patient P and one fluid conduit 25, 26, 27 for treatment fluid;
5—all fluid conduits 25, 26, 27 for additional fluids, which may be an replacement solution before entering into a corresponding circuit 6, 7, 8 or additional fluids directly supplied to the patient P;
6—two fluid conduits 25, 26, 27 for blood, systemic and treated, and one fluid conduit 25, 26, 27 for treatment fluid;
7—one fluid conduit 25, 26, 27 for treatment fluid to be supplied to the blood treatment unit 2, one fluid conduit 25, 26, 27 for treatment unit to be supplied directly to the patient P by passing the treatment unit 2 and one fluid conduit 25, 26, 27 for blood;
8—one fluid conduit 25, 26, 27 for treatment fluid to be supplied to the blood treatment unit 2, one fluid conduit 25, 26, 27 for treatment unit to be supplied directly to the patient P by passing the treatment unit 2 and one fluid conduit 25, 26, 27 for one additional fluid, which may be an replacement solution before entering into a corresponding circuit 6, 7, 8 or one additional fluid directly supplied to the patient P.

In a similar way, in order to allow the connection between the fluid conduits 25, 26, 27 to the lines 7a, 7b of the extracorporeal blood circuit 7 or the dialysis line 8a of the treatment fluid circuit 8 or the replacement lines 12, 13, 14a, 14b of the infusion circuit or any distinct additional fluid circuit, it is sufficient to connect the corresponding connectors 28c, 29c, 30c, 31c, 32c, 33c of the corresponding inlet tubes 28, 29, 30 and outlet tube 31, 32, 33 to the specific connection points of such circuits or lines.

Naturally, if the systemic blood or the treated blood are infused with at least one infusion solution before entering the corresponding fluid conduit 25, 26, 27 of the fluid warming/cooling bag 24 the temperature of the infused blood is controlled and regulated by the temperature control device 15. Similarly, if the treatment fluid is infused with at least one replacement solution before entering the corresponding fluid conduit 25, 26, 27 of the fluid warming/cooling bag 24 the temperature of the infused blood is controlled and regulated by the temperature control device 15.

Advantageously, at least one deaeration/filtration device, not shown, is operatively connected to or is configured to be connected to at least one of the outlet ports 25b, 26b, 27b of the fluid conduits 25, 26, 27 of the fluid warming/cooling bag 24. Optionally a deaeration/filtration device is operatively connected to or is configured to be connected to each outlet ports of the fluid conduits 25, 26, 27 of the fluid warming/cooling bag 24 or one a deaeration/filtration device is operatively connected to or is configured to be connected to all fluid conduits 25, 26, 27 of the fluid warming/cooling bag 24, like the shown air trapping device 10.

Considering the apparatus 1 shown in FIG. 1, it has to be noted that the blood treatment unit 2, the fluid circuit 6, even if it includes the extracorporeal blood circuit 7 and the treatment fluid circuit 8 and only one of such circuits, the fluid warming/cooling bag 24 coupled to the fluid circuit 6 form and define a disposable extracorporeal blood treatment tubing set which may be easily packaged into a corresponding useful and compact packaging case. In the packaged configuration, the fluid conduits 25, 26, 27 of the fluid warming/cooling bag 24 are dry and empty being such bag flat. The package is sealed and properly sterilized.

1-16. (canceled)

17. An extracorporeal blood treatment tubing set for an extracorporeal blood treatment apparatus, the extracorporeal blood treatment tubing set comprising:
a blood treatment unit;
a fluid circuit coupled to the blood treatment unit, the fluid circuit including:
an extracorporeal blood circuit with a blood withdrawal line connected to an inlet port of the blood treatment unit and a blood return line connected to an outlet port of the blood treatment unit, and
a treatment fluid circuit operatively connected to the extracorporeal blood circuit and/or to the blood treatment unit; and
a fluid warming/cooling bag coupled to the fluid circuit and comprising:
a first inlet port,
a first outlet port,
a first fluid conduit configured for putting the first inlet port in fluid communication with the first outlet port,
a second inlet port,
a second outlet port, and
a second fluid conduit configured for putting the second inlet port in fluid communication with the second outlet port, the first fluid conduit and the second fluid conduit being fluidically separated from each other.

18. The extracorporeal blood treatment tubing set according to claim 17, wherein at least one of the first fluid conduit and the second fluid conduit of the fluid warming/cooling bag is in fluid communication with the extracorporeal blood circuit of the fluid circuit to be supplied with blood or blood infused with at least an infusion solution, and
wherein another one of the first fluid conduit and the second fluid conduit of the fluid warming/cooling bag is in fluid communication with the treatment fluid circuit of the fluid circuit.

19. The extracorporeal blood treatment tubing set according to claim 17, wherein the fluid warming/cooling bag comprises:
a third inlet port;
a third outlet port; and
a third fluid conduit configured for putting the third inlet port in fluid communication with the third outlet port, the third fluid conduit being fluidically separated from the first and the second fluid conduits.

20. The extracorporeal blood treatment tubing set according to claim 17, wherein each of the first and second fluid conduits of the fluid warming/cooling bag includes a corresponding elongated fluid channel defining a flow direction for a corresponding fluid from the corresponding inlet port to the outlet port, wherein each of the first and second fluid conduits comprises:
an inlet portion in direct communication with the corresponding inlet port;
an outlet portion in direct communication with the corresponding outlet port; and
an intermediate portion connecting the corresponding inlet portion to the corresponding outlet portion and developing along a development direction, wherein the corresponding intermediate portion has a constant cross-sectional dimension.

21. The extracorporeal blood treatment tubing set according to claim 20, wherein:
the inlet portion of each of the first and second fluid conduits has a cross-sectional dimension increasing moving from the corresponding inlet port to the corresponding intermediate portion;
the outlet portion of the first and second fluid conduits have a cross-sectional dimension decreasing moving from the corresponding intermediate portion to the corresponding outlet port, and
the intermediate portion of the first and second fluid conduits includes:
two corresponding parallel and rectilinear main tracts, and
a corresponding bend tract connecting the corresponding main tracts, the bend tract being arched or circular.

22. The extracorporeal blood treatment tubing set according to claim 21, wherein the two main tracts and the bend tract of each conduit are respectively parallel to the two main tracts and the bend tract of any other fluid conduit of the fluid warming/cooling bag, wherein the intermediate portion of each of the first and second fluid conduits has a U-shape or a C-shape configuration.

23. The extracorporeal blood treatment tubing set according to claim 21, wherein one fluid conduit of the first and second fluid conduits of the fluid warming/cooling bag is placed among the two main tracts and the bend tract of at least one other fluid conduit of the first and second fluid conduits of the fluid warming/cooling bag.

24. The extracorporeal blood treatment tubing set according to claim 17, wherein the inlet port and the outlet port of each of the first and second fluid conduits are placed on a same side of the fluid warming/cooling bag, each of the first and second fluid conduits having only one inlet port and one outlet port.

25. The extracorporeal blood treatment tubing set according to claim 17, wherein the fluid warming/cooling bag comprises:
an inlet tube for each of the first and second fluid conduits, the inlet tube including:
a corresponding constrained end connected to the corresponding inlet port of the respective first and second fluid conduits, and
a free end, opposite to the constrained end, provided with a corresponding connector for connection to a corresponding tube of the extracorporeal blood treatment tubing set of the extracorporeal blood treatment apparatus or a corresponding tube of the fluid circuit of the extracorporeal blood treatment apparatus; and
an outlet tube for each of the first and second fluid conduits, the outlet tube having:
a corresponding constrained end connected to the corresponding outlet port of the respective first and second fluid conduits, and
a free end, opposite to the constrained end, provided with a connector for connection to a corresponding tube of the extracorporeal blood treatment tubing set of the extracorporeal blood treatment apparatus or a corresponding tube of the fluid circuit of the extracorporeal blood treatment apparatus.

26. The extracorporeal blood treatment tubing set according to claim 25, wherein:
the connector of the free end of the inlet tube of each of the first and second fluid conduits is a male connector or a male Luer connector, and
the connector of the free end of the outlet tube of each of the first and second fluid conduits is a female connector or a female Luer connector.

27. The extracorporeal blood treatment tubing set according to claim 17, wherein the fluid warming/cooling bag comprises at least one marking to provide a distinction of each of the first and second fluid conduits with respect to the other fluid conduit.

28. The extracorporeal blood treatment tubing set according to claim 27, wherein each marking comprises one or more of:
a color marking, including as an identifying color placed on the corresponding inlet tube and/or on the corresponding outlet tube and/or on the corresponding fluid conduit,
a dimensional marking, including a length of the corresponding inlet tube and/or a length of the corresponding outlet tube, and
the external appearance of the connector of the corresponding inlet tube and/or the connector of the corresponding outlet tube.

29. The extracorporeal blood treatment tubing set according to claim 17, wherein the fluid warming/cooling bag is flat and flexible and is made of two sealed plastic films, the sealing defining each fluid conduit.

30. The extracorporeal blood treatment tubing set according to claim 17, wherein the fluid warming/cooling bag has a tab protruding on a side opposite to the side where the inlet port and the outlet port of each of the first and second fluid conduits are placed, wherein the tab is positioned aside with respect to a median longitudinal axis of the fluid warming/cooling bag, thereby giving the fluid warming/cooling bag an asymmetric shape.

31. The extracorporeal blood treatment tubing set according to claim 17, wherein:
one fluid conduit of the first and second fluid conduits of the fluid warming/cooling bag is in fluid communication with the treatment fluid circuit of the fluid circuit to be supplied with a treatment fluid or an infusion solution, and
another fluid conduit of the first and second fluid conduits of the fluid warming/cooling bag is in fluid communication with the treatment fluid circuit of the fluid circuit to be supplied with a treatment fluid or an infusion solution.

32. The extracorporeal blood treatment tubing set according to claim 17, wherein the fluid warming/cooling bag is made of thermoplastic polyurethane.

33. The extracorporeal blood treatment tubing set according to claim 17, wherein the extracorporeal blood treatment tubing set is disposable.

34. The extracorporeal blood treatment tubing set according to claim 17, wherein a cross-sectional dimension of the first fluid conduit is different from a cross-sectional dimension of the second fluid conduit.

35. The extracorporeal blood treatment tubing set according to claim 17, wherein the fluid warming/cooling bag is flat and the first and second fluid conduits are configured to assume a three-dimensional shape as fluid swells one or both the first and second fluid conduits.

36. An extracorporeal blood treatment tubing set for an extracorporeal blood treatment apparatus, the extracorporeal blood treatment tubing set comprising:
a blood treatment unit;
a fluid circuit coupled to the blood treatment unit, the fluid circuit including:
an extracorporeal blood circuit with a blood withdrawal line connected to an inlet port of the blood treatment unit and a blood return line connected to an outlet port of the blood treatment unit, and
a treatment fluid circuit operatively connected to the extracorporeal blood circuit and/or to the blood treatment unit; and
a fluid warming/cooling bag coupled to the fluid circuit and comprising:
a first inlet port,
a first outlet port,
a first fluid conduit configured for putting the first inlet port in fluid communication with the first outlet port,
a second inlet port,
a second outlet port, and
a second fluid conduit configured for putting the second inlet port in fluid communication with the second outlet port, the first fluid conduit and the second fluid conduit being fluidically separated from each other,
wherein the fluid warming/cooling bag is flat and flexible and is made of two sealed flexible plastic films, the sealing defining each of the first and second fluid conduits, and
wherein the first conduit and second fluid conduits are flat when no fluid flows therein and configured to assume a three-dimensional shape as fluid swells the first and second fluid conduits.

37. An extracorporeal blood treatment apparatus comprising:
a blood treatment unit;
a fluid circuit coupled to the blood treatment unit, the fluid circuit including:
an extracorporeal blood circuit with a blood withdrawal line connected to an inlet port of the blood treatment unit and a blood return line connected to an outlet port of the blood treatment unit, and
a treatment fluid circuit operatively connected to the extracorporeal blood circuit and/or to the blood treatment unit;
a fluid pump coupled to the fluid circuit to determine a fluid flow inside the fluid circuit;
a controller configured to control the fluid pump;
a fluid temperature control device coupled to the fluid circuit and comprising:
a receptacle, and
a heat exchange region; and
a fluid warming/cooling bag configured to be accommodated in the receptacle of the fluid temperature control device, the fluid warming/cooling bag being coupled to the fluid circuit and comprising:
a first inlet port,
a first outlet port,
a first fluid conduit configured for putting the first inlet port in fluid communication with the first outlet port,
a second inlet port,
a second outlet port, and
a second fluid conduit configured for putting the second inlet port in fluid communication with the second outlet port, the first fluid conduit and the second fluid conduit being fluidically separated from each other,
wherein, when the fluid warming/cooling bag is accommodated in the receptacle, and
wherein the fluid temperature control device is configured to effect a heat exchange with two separate fluids inside each of the first and second fluid conduits of the warming/cooling bag through the corresponding fluid conduit for simultaneously determining a heat exchange with the two separate fluids inside the first and second fluid conduits of the warming/cooling bag.
